(19) United States
(12) Patent Application Publication
(10) Pub. No.: US 2022/0387357 A1
(43) Pub. Date: Dec. 8, 2022

(54) Method of Dosing a Pain Therapeutic

(71) Applicant: Cytometix, Inc., Wauwatosa, WI (US)

(72) Inventor: Lane Brostrom, Eden, UT (US)

(21) Appl. No.: 17/730,375

(22) Filed: Apr. 27, 2022

Related U.S. Application Data
(60) Provisional application No. 63/196,924, filed on Jun. 4, 2021.

(51) Int. Cl. A61K 31/16; A61P 9/06; A61K 9/00; A61K 9/08

(52) U.S. Cl. CPC A61K 31/16; A61P 9/06; A61K 9/0019; A61K 9/08

(57) ABSTRACT

A method for minimizing tachycardia in a subject treated for pain and experiencing tachycardia after being treated with the compound having the structure:

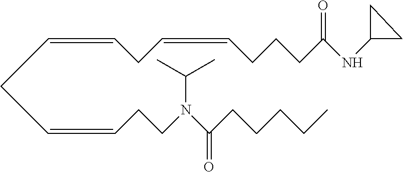 [SMILES: CCCCCC(=O)N(CCC=C=CCCCC=C=CCC=C=CCCCC(=O)NC1CC1)C(C)C]

An effective amount of the compound is administered to a subject, where the effective amount treats pain and minimizes tachycardia in said subject experiencing tachycardia after being administered said compound.

### CROSS-REFERENCE TO RELATED APPLICATIONS

This application claims the benefit of U.S. Provisional Application No. 63/196,924, filed Jun. 4, 2021, hereby incorporated by reference.

### STATEMENT REGARDING FEDERALLY FUNDED RESEARCH AND DEVELOPMENT

Not Applicable.

### FIELD OF THE INVENTION

This invention relates to the treatment of pain. In particular this invention is directed to a method of dosing a pain therapeutic while reducing tachycardia.

### BACKGROUND OF THE INVENTION

The pain pathway begins in the periphery with nociceptors that innervate skin, muscle, tendon or bone targets. Activated or sensitized nociceptors transmit noxious information to the spinal dorsal horn where spinal neurons then transmit information to rostral centers in the thalamus, reticular formation and midbrain. Other neurons carry the information to the somatosensory cortex where pain is interpreted. Nociceptive information transmitted through the spinal cord is heavily modulated by central neurons whose axons descend from the midbrain and other rostral areas to the spinal cord, and these descending pathways can be either inhibitory or faciliatory.

Neurons contain a variety of voltage-gated ion channels. The voltage-gated K+ and Na+ channels regulate the excitability of neuronal cells and play a crucial role in setting the perceptual threshold of pain. The ability to modulate the activity of K+ or Na+ ion channels in neuronal cells is important for regulating the transmission of pain signals.

Epoxyeicosatrienoic acids (EETs) are produced from arachidonic acid via cytochrome P450 (CYP) epoxygenases. EETs regulate inflammation, angiogenesis, cellular proliferation, ion transport and steroidogenesis. In many issues, EET levels are regulated, inter alia, through their metabolism to vic-diols (vic-dihydroxyeicosatrienoic acids; DiHETrE) via the enzyme soluble epoxide hydrolase (EPHX2).

While some types of pain are effectively managed with opioids such as morphine or non-steroidal anti-inflammatory drugs (NSAIDS) such as aspirin or ibuprofen, opioids and NSAIDS both have numerous undesirable side effects. For instance, opioids frequently cause dependence and withdrawal problems in users. The use of opioids in the management of pain is further limited due to impairment of Na+/K+-ATPase activity after opiate treatment, a possible mechanism of tolerance/addiction. Similarly, NSAIDS can cause hypertension, ulcer perforation, upper gastrointestinal bleeding and even death in severe cases.

Acetaminophen is one of the most widely used drugs in the world for treatment of pain and fever, and probably the most commonly prescribed medicine in children. Over 600 products contain acetaminophen including OTC pain, cold and flu remedies and prescription medications like Vicodin. It has a unique position among analgesic drugs. Unlike NSAIDs, it is considered an ineffective anti-inflammatory, but does not produce gastrointestinal damage or untoward cardio-renal effects; unlike opiates, it is ineffective in pain arising from smooth muscle spasm but has no depressant effect on respiration. The acetaminophen metabolite that produces analgesia is AM40413 now known to provide analgesia through CB1 and TRPV1 receptors. Unfortunately, acetaminophen is toxic in high doses and is responsible for the majority of the acute liver failure cases in the United States. NAPQI is the molecule largely believed to be responsible for liver failure.

Accordingly, a need exists for improved analgesic treatment that avoids the above-mentioned side effects but provides an effective and safe treatment for pain. CMX-020 is a rapidly acting intravenous analgesic that chemically is a close structural analog of a set of natural lipid mediators derived from arachidonic acid that are used to control pain. The chemical structure and receptor activity of CMX-020 are also common to those of AM404, the active metabolite of acetaminophen. Side-by-side mice studies comparing the analgesic efficacy of CMX-020 and AM404 demonstrate both compounds share the highest level of analgesic efficacy in the acute pain tail-flick assay similar to opioids. AM404 also has demonstrated anticonvulsant action mediated through CB1 receptors. CMX-020 intravenous investigational product is a solution containing CMX-020 drug substance (3 mg/mL), ascorbic acid (0.0135 mg/mL), hydroxypropyl-β-cyclodextrin (HBCD) (200 mg/mL) in a diluent composed of 35% phosphate buffered saline (PBS)/65% sterile water, packaged and supplied in 25 mL vials containing 20.5 mL of the investigational product. HBCD is used in the formulation as a solubilizer and stabilizer. Observations from nonclinical studies have shown that administration of CMX-020 results in analgesia equivalent to that achieved by morphine.

In preclinical trials, CMX-020 has been shown to effectively reduce the pain response in several animal models of pain, including models of acute pain, visceral pain, fibromyalgia, diabetic peripheral neuropathy, sciatica, chemotherapy induced pain, and osteoarthritis. Previously conducted trials with both intravenous and oral formulations of CMX-020 have demonstrated the safety of CMX-020 when administered to humans at several dose levels and have indicated efficacy in a proof-of-concept trial in sciatic pain. Nevertheless, CMX-020 may produce adverse side effects when given at improper dosages. Methods of administrating an effective amount of the compound CMX-020 to produce an analgesia effective, while still being within a safe and tolerable dose, is both necessary and desirable.

### SUMMARY OF THE INVENTION

In a subset of subjects treated with CMX-020, the compound (shown below) may cause tachycardia. This invention is directed towards minimizing tachycardia in the subset of subjects treated for pain that experience the adverse event, tachycardia.

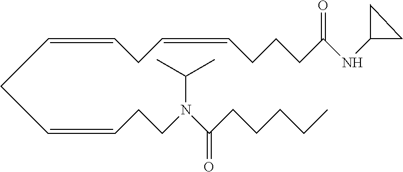 [SMILES: CCCCCC(=O)N(CCC=C=CCCCC=C=CCC=C=CCCCC(=O)NC1CC1)C(C)C]

Accordingly, the invention encompasses novel methods of minimizing tachycardia in a subject being treated for pain with the compound CMX-020, comprising administering to a subject experiencing tachycardia during pain treatment an effective amount of the compound CMX-020, wherein said effective amount treats pain and minimizes tachycardia in said subject.

In one aspect of the invention, the compound CMX-20 is a dosage form in an intravenous solution.

In terms of an effective amount to minimize tachycardia in a subject treated with CMX-020, CMX-020 is administered at about 0.02 mg/kg to about 0.32 mg/kg, at about 0.04 mg/kg to about 0.24 mg/kg, or at about 0.16 mg/kg to about 0.32 mg/kg over about 15 to about 30 minutes to the subject.

In another aspect of the invention, the effective amount of CMX-020 is administered at about 0.02 mg/kg, at about 0.04 mg/kg, at about 0.08 mg/kg, at about 0.16 mg/kg, or about 0.24 mg/kg over about 15 to about 30 minutes to the subject.

In still yet another aspect of the invention, said effective amount of the compound CMX-020 is administered to the subject at a rate over about 30 minutes to achieve a plasma concentration of CMX-020 of about 1420 ng/ml to about 1525 ng/ml in the subject. The compound CMX-020 is administered to the subject at a steady rate. The steady rate of administration is about 0.048 mg/kg/hr to about 0.64 mg/kg/hr. The plasma concentration of CMX-020 is maintained by steady rate of administration in the subject for at least 6 hours.

These particular objects and advantages may apply to only some embodiments falling within the claims and thus do not define the scope of the invention.

### DETAILED DESCRIPTION OF THE INVENTION

Before the present materials and methods are described, it is understood that this invention is not limited to the particular methodology, protocols, materials, and reagents described, as these may vary. It is also to be understood that the terminology used herein is for the purpose of describing particular embodiments only and is not intended to limit the scope of the present invention which will be limited only by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described.

The disclosed compound formulas and structures can in some cases vary between neutral, acid, and/or basic salt forms, depending on the surrounding environment, and such forms may be used interchangeably herein. As a non-limiting example, a primary amine moiety on a compound may be interchangeably designated as −NH₂ or as NH₃⁺. Furthermore, a given compound may have equivalent resonance structures, which may be used interchangeably herein.

All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

As used herein, the term “administering” refers to bringing a subject, tissue, organ or cells in contact with the compound CMX-020 as described in this disclosure. In certain embodiments, the present invention encompasses administering the compounds useful in the present invention to a patient or subject. A “subject,” “patient” and “individual,” used equivalently herein, refers to a mammal, preferably a human.

As used herein, the terms “effective amount” and “therapeutically effective amount” refer to the quantity of active therapeutic agents sufficient to yield a desired therapeutic response without undue adverse side effects such as toxicity, irritation, or allergic response. The specific “effective amount” will, obviously, vary with such factors as the particular condition being treated, the physical condition of the patient, the duration of the treatment, the nature of concurrent therapy (if any), and the specific formulations employed. In this case, an amount would be deemed therapeutically effective if it results in an analgesic effective in the subject to provide a safe method of pain treatment. The optimum effective amounts can be readily determined by one of ordinary skill in the art using routine experimentation.

An “effective amount” includes administering CMX-020 at about 0.02 mg/kg to about 0.32 mg/kg, at about 0.04 mg/kg to about 0.24 mg/kg, or at about 0.16 mg/kg to about 0.32 mg/kg over about 15 to about 30 minutes to the subject. In still yet another “effective amount” of CMX-020 includes administering CMX-020 at about 0.02 mg/kg, at about 0.04 mg/kg, at about 0.08 mg/kg, at about 0.16 mg/kg, or about 0.24 mg/kg over about 15 to about 30 minutes to the subject. An effective amount of the compound CMX-020 is a plasma concentration of CMX-020 of about 1420 ng/ml to about 1525 ng/ml in the subject when CMX-020 is administered to a subject at a steady rate over about 30 minutes.

The disclosure also provides pharmaceutical compositions comprising one or more compounds of this invention in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. It is also envisioned that the compounds of the present invention may be incorporated into transdermal patches designed to deliver the appropriate amount of the drug in a continuous fashion.

For preparing solid compositions such as tablets, the principal active ingredient is mixed with a pharmaceutically acceptable carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture for a compound of the present invention, or a pharmaceutically acceptable salt thereof When referring to these preformulation compositions as homogeneous, it is meant that the active ingredient is dispersed evenly throughout the composition so that the composition may be easily subdivided into equally effective unit dosage forms such as tablets, pills and capsules. The tablets or pills can be coated or otherwise compounded to provide a dosage affording the advantage of prolonged action. For example, the tablet or pill can comprise an inner dosage and an outer dosage component, the latter being in the form of an envelope over the former. The two components can be separated by an enteric layer which, serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium caboxymethylcellulose, methylcellulose, polyvinylpyrrolidone or gelatin.

The following examples are, of course, offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

### Example 1: Safely Evaluation

Tachycardia was the most common adverse event (AE) in this study, reported in 5 of 48 subjects (10%) following active treatment and in no subjects following placebo; all of the treatment-emergent AEs (TEAEs) of tachycardia were deemed related to study treatment. Tachycardia was only reported in subjects who received CMX-020, and only at the three highest doses (0.32 mg/kg and 0.24 mg/kg 15 minutes infusion and 0.32 mg/kg 30 minute infusion).

The Toxicity Grading Scale for Healthy Adult and Adolescent Volunteers Enrolled in Preventative Vaccine Clinical Trials criteria for tachycardia are defined as follows:

Mild (Grade 1): 101-116 bpm

Moderate (Grade 2): 117-130 bpm

Severe (Grade 3): >130 bpm

In this study, tachycardia was dose limiting at a level >130 bpm. The tachycardia TEAEs in this study are summarized in Table 1.

TABLE 1
Tachycardia TEAEs with Time to Down-Grade to “Mild”

| | Subject | TEAE | Peak | Time to Down-Grade |
|---|---|---|---|---|
| Cohort | Number | Coding | Pulse | to “Mild” |
| 5 | 0033 | Moderate* | 138 bpm | 15 min |
| 5 | 0036 | Mild | 110 bpm | — |
| 6 | 0045 | Moderate* | 156 bpm | 10 min |
| 6 | 0048 | Mild | 109 bpm | — |
| 8 | 0057 | Mild | 110 bpm | — |

*“Moderate” because short-lived in a hospital setting

There were two TEAEs of tachycardia that occurred in Cohort 5 and Cohort 6 that were dose-limiting (>130 bpm). As shown in the table above, these TEAEs decreased in severity to “mild” within 15 minutes, and 10 minutes, respectively.

There were 3 subjects with vital signs measurements assessed as clinically significant as follows:

Subject 0033 (CMX-020 0.32 mg/kg 15-minute infusion) had a peak heart rate of 138 bpm at 16 minutes following the start of treatment administration, assessed as abnormal clinically significant. This subject's heart rate decreased to 113 bpm 15 minutes following the peak heart rate, when it was assessed as not clinically significant. This event was reported as a TEAE of tachycardia (moderate, probably related).

Subject 0036 (CMX-020 0.32 mg/kg 15-minute infusion) had a peak heart rate of 110 bpm 16 minutes following the start of dose administration, assessed as abnormal clinically significant. This subject's heart rate decreased to 101 bpm 15 minutes following the start of treatment administration, when it was assessed as not clinically significant. This event was reported as a TEAE of tachycardia (mild, probably related).

Subject 0045 (CMX-020 0.24 mg/kg 15-minute infusion) had an elevated heart rate (156 bpm) at 11 minutes following the start of dose administration, assessed as abnormal clinically significant. This subject's heart rate decreased to 109 bpm 10 minutes following the start of treatment administration, when it was assessed as not clinically significant. This event was reported as a TEAE of tachycardia (moderate, probably related). Study treatment was discontinued in this subject following 6.5 mL of a 10 mL infusion (total dose received 0.156 mg/kg). The subject was very anxious prior to, and during, CMX-020 administration.

Vital signs measurements associated with TEAEs were as follows:

Subject 0048 (CMX-020 0.24 mg/kg 15-minute infusion) reported a TEAE of tachycardia (mild, probably related) 11 minutes following start of treatment administration. This subject's heart rate peaked at 109 bpm 16 minutes following start of treatment administration. This subject's heart rate decreased to 103 bpm 15 minutes following the peak heart rate.

Subject 0057 (CMX-020 0.32 mg/kg 30-minute infusion) reported a TEAE of tachycardia (mild, probably related) 16 minutes following start of treatment administration. This subject's heart rate peaked at 110 bpm 20 minutes following start of treatment administration. This subject's heart rate decreased to 97 bpm 20 minutes following the peak heart rate.

There were a higher number of elevated heart rate assessed as clinically significant in the CMX-020 0.32 mg/kg 15-minute infusion group and CMX-020 0.24 mg/kg 15-minute infusion group compared with the other CMX-020 dose levels and compared with subjects who received placebo. Among cohorts 5 and 6 with 15-minute infusions, the peak in elevated heart rate occurred between 11-16 minutes following the start of treatment administration. Among cohorts 7 and 8 with 30-minute infusions, the peak in elevated heart rate occurred at approximately 30 minutes.

While this invention has been described in conjunction with the various exemplary embodiments outlined above, various alternatives, modifications, variations, improvements and/or substantial equivalents, whether known or that are or may be presently unforeseen, may become apparent to those having at least ordinary skill in the art. Accordingly, the exemplary embodiments according to this invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents (including enantiomers) of these exemplary embodiments. All technical publications, patents and published patent applications cited herein are hereby incorporated by reference in their entirety for all purposes.

### REFERENCES

1. Högestätt ED, Jönsson BA, Ermund A, Andersson DA, Björk H, Alexander JP, Cravatt BF, Basbaum AI, Zygmunt PM. Conversion of acetaminophen to the bioactive N-acylphenolamine AM404 via fatty acid amide hydrolase-dependent arachidonic acid conjugation in the nervous system. J Biol Chem. 2005;280:31405-31412. Epub 2005 Jun. 29.

2. Deshpande LS, DeLorenzo RJ. Acetaminophen inhibits status epilepticus in cultured hippocampal neurons. Neuroreport. 2011 Jan. 5; 22(1): 15-18.

3. Jasinski DR, Krishnan S. Abuse liability and safety of oral lisdexamfetamine dimesylate in individuals with a history of stimulant abuse. J Psychopharmacol. 2009; 23:419-427.

4. Parasrampuria DA, Schoedel KA, Schuller R, et al. Do formulation differences alter abuse liability of methylphenidate? J Clin Psychopharmacol. 2007; 27:459-467.

5. Terashvili, M, Tseng LF, Wu HE, et al. Antinociception produced by 14, 15-epoxyeicosatrienoic acid is mediated by the activation of β-endorphin and met-enkephalin in the rat ventrolateral periaqueductal gray. J Pharmacol Exp Ther 2008; 326(2):614-622.

What is claimed is:

1. A method for minimizing tachycardia in a subject treated for pain with the compound CMX-020, comprising administering to a subject experiencing tachycardia during pain treatment an effective amount of the compound CMX-020, wherein said effective amount treats pain and minimizes tachycardia in said subject.

2. The method of claim 1, wherein said compound CMX-020 is a dosage form in an intravenous solution.

3. The method of claim 1, wherein said compound CMX-020 is intravenously administered to said subject.

4. The method of claim 1, wherein the compound CMX-020 is administered at about 0.02 mg/kg to about 0.32 mg/kg over about 15 to about 30 minutes to the subject.

5. The method of claim 1, wherein the compound CMX-020 is administered at about 0.04 mg/kg to about 0.24 mg/kg over about 15 to about 30 minutes to the subject.

6. The method of claim 1, wherein the compound CMX-020 is administered at about 0.16 mg/kg to about 0.32 mg/kg over about 15 to about 30 minutes to the subject.

7. The method of claim 1, wherein the compound CMX-020 is administered at about 0.02 mg/kg over about 15 to about 30 minutes to the subject.

8. The method of claim 1, wherein the compound CMX-020 is administered at about 0.04 mg/kg over about 15 to about 30 minutes to the subject.

9. The method of claim 1, wherein the compound CMX-020 is administered at about 0.08 mg/kg over about 15 to about 30 minutes to the subject.

10. The method of claim 1, wherein the compound CMX-020 is administered at about 0.16 mg/kg over about 15 to about 30 minutes to the subject.

11. The method of claim 1, wherein the compound CMX-020 is administered at about 0.24 mg/kg over about 15 to about 30 minutes to the subject.

12. The method of claim 1, wherein said effective amount of the compound CMX-020 is administered to the subject at a rate over about 30 minutes to achieve a plasma concentration of CMX-020 of about 1420 ng/ml to about 1525 ng/ml in the subject.

13. The method of claim 12, wherein said effective amount of the compound CMX-020 is administered to the subject at a steady rate.

14. The method of claim 13, wherein said steady rate is about 0.48 mg/kg/hr to about 0.64 mg/kg/hr.

15. The method of claim 12, wherein the plasma concentration of CMX-020 is maintained by steady rate of administration in the subject for at least about 6 hours.

16. A compound having the structure 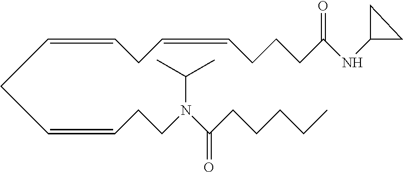 [SMILES: CCCCCC(=O)N(CCC=C=CCCCC=C=CCC=C=CCCCC(=O)NC1CC1)C(C)C]
for use in minimizing tachycardia in a subject experiencing tachycardia after being administered said compound for treatment of pain.

17. A compound having the structure 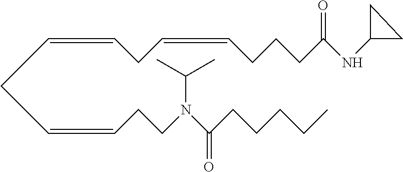 [SMILES: CCCCCC(=O)N(CCC=C=CCCCC=C=CCC=C=CCCCC(=O)NC1CC1)C(C)C]
for use in a method according to claim 1.

18. An intravenous solution comprising an effective amount of a compound having the structure 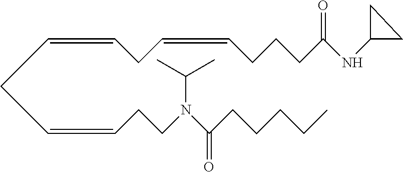 [SMILES: CCCCCC(=O)N(CCC=C=CCCCC=C=CCC=C=CCCCC(=O)NC1CC1)C(C)C]
for use in minimizing tachycardia in a subject experiencing tachycardia as a result of being administered said intravenous solution for treatment of pain.

19. An intravenous solution comprising an effective amount of a compound having the structure 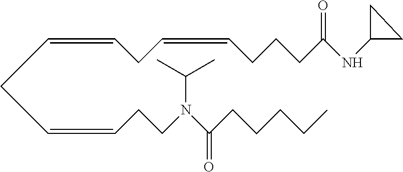 [SMILES: CCCCCC(=O)N(CCC=C=CCCCC=C=CCC=C=CCCCC(=O)NC1CC1)C(C)C]
for use in a method according to claim 1.
